(19) United States
(12) Patent Application Publication
(10) Pub. No.: US 2018/0078200 A1
(43) Pub. Date: Mar. 22, 2018

(54) APPARATUS, SYSTEMS, AND METHODS FOR MONITORING ADHERENCE TO A TREATMENT PROTOCOL AND NEED FOR PRODUCT REFILL

(71) Applicant: N-2 Medical Solutions, LLC, Winston-Salem, NC (US)

(72) Inventors: Andrew Bowline, Winston-Salem, NC (US); Scott Coldagelli, Raleigh, NC (US); Adam Oakman, Austin, TX (US)

(21) Appl. No.: 15/565,178

(22) PCT Filed: Apr. 11, 2016
(86) PCT No.: PCT/US16/26899

§ 371 Date: Oct. 9, 2017

Related U.S. Application Data
(60) Provisional application No. 62/145,238, filed on Apr. 9, 2015.

(51) Int. Cl. A61B 5/00; A61J 7/04; A61J 1/03; G06F 19/00

(52) U.S. Cl. CPC A61B 5/4833; A61J 7/0418; A61J 1/035; A61J 7/0481; G06Q 10/087; G06F 19/3456; A61J 2200/30; A61J 2200/74; A61J 7/0436

(57) ABSTRACT

Embodiments of the present invention provide apparatus, methods and systems that are advantageous for multiple uses, including monitoring a user's, e.g., a patient's, 25 adherence to a treatment protocol.

### REFERENCE TO RELATED APPLICATION

This application claims priority to U.S. Provisional Patent Application No. 62/145,238, entitled “Apparatus, Systems, And Methods For Monitoring Adherence To A Treatment Protocol And Need For Product Refill,” filed Apr. 9, 2015, the entirety of which is hereby incorporated by reference herein.

### FIELD OF THE INVENTION

This patent relates in some aspects to the medical field. Disclosed exemplary embodiments include apparatus, systems and methods for monitoring adherence to a treatment protocol related to a consumable product, and further include new ways to capture, collect, and use adherence data. Product refill needs are also monitored.

### BACKGROUND OF THE INVENTION

Many patients do not adhere to a treatment protocol as planned, e.g., as prescribed by their physician or health care provider. This problem with adherence is well-documented and creates situations where the effectiveness of a prescribed treatment cannot be properly evaluated, either by the patient, the patient's physician, other health care providers, insurers, government regulatory agencies, or drug companies. The estimated annual costs of nonadherence are billions of dollars and over a hundred thousand deaths in the United States alone.

### SUMMARY OF THE INVENTION

Embodiments of the present invention provide apparatus, methods and systems that are advantageous for multiple uses, including monitoring a user's, e.g., a patient's, adherence to a treatment protocol.

In an aspect, the present invention provides an apparatus that includes a vessel or enclosure for holding a product, and/or plurality of products, including, but not limited to a pharmaceutical product, vitamin, nutrition aid, and/or the like; and a measuring apparatus (i.e., measurement means) capable of determining the amount, quantity, mass, weight, volume, capacitance, opacity, light absorption or reflection, or similar physical and/or chemical characteristic of the product in the enclosure and also capable of detecting a change in that characteristic. The apparatus may further comprise measuring and/or computing components, such as a data collector for collecting data relating to the characteristic, and/or a change in the characteristic; and/or memory for retaining the data, or other details relating to the characteristic. The apparatus may further comprise a processor; first communication means for transmitting electrical signals and/or data bits among the component parts of the apparatus; and second communication means for communicating the data to an external server. The apparatus may still further comprise components typical of an electrical apparatus such as a power supply; a transformer; a display; switches (e.g. a power switch); an AC/DC converter; an AC connection; a power cord; a timer; a clock; an alarm; an audio and/or visual signal, with generating means for each; and/or an antenna. The foregoing list, and description, is not exhaustive and an apparatus of the present invention may include additional features and/or components.

In an embodiment, operation of the apparatus of the present invention comprises detecting a change in the characteristic of the product, such as a change in the overall weight or capacitance of the product in the vessel or enclosure. Adherence by the user of the apparatus, such as a patient, to a treatment protocol may be monitored by monitoring the change in the characteristic of the product. The adherence may be communicated to the user, e.g., through a display, and/or may be communicated in real-time or asynchronously to a receiving application or a human monitor, that may be located remotely, such as a health care provider, for monitoring adherence. The remote communication may be data communicated to a database on a server that is monitored by the monitor.

For example, a non-limiting exemplary use of the apparatus could be by a user desiring, or prescribed, to take 81 mg of aspirin daily, for example as possible prevention for heart attack and/or stroke. The enclosure of the apparatus would include a plurality of 81 mg aspirin tablets. When the user removes an aspirin tablet, a change in the overall weight of aspirin is detected and communicated to the user and a remote monitor. Adherence to the treatment protocol of one aspirin tablet per day is thereby monitored. As will be appreciated, in embodiments the apparatus may include an audio and/or visual signal to alert the user of the need to take the aspirin tablet that day.

In a further aspect, the present invention provides systems for monitoring adherence to a treatment protocol. In some embodiments a system for monitoring adherence comprises an apparatus of the present invention, as described above and herein. The system may further comprise one or more of the following components: a component for transmitting data, a component for receiving data, a component for processing data, a component for displaying data and/or determinations made by processing the data, and associated software. Some embodiments include a component for transmitting an alert to a designated party in case a specified result is determined. One or more of these components may be located remotely from the apparatus and accessed, for example, through a communications protocol.

In some embodiments, systems as described above and herein may be used to monitor need for a product refill. The product may be a prescription or non-prescription pharmaceutical product, or another type of consumable product. In such embodiments, a user inputs a baseline level of a characteristic to trigger ordering of a refill (e.g., when the contents decrease to 20% of full weight). In some such embodiments, the determination of a triggering baseline characteristic generates an alert to a designated party, such as the user, a pharmacist, a health care provider and/or other product supplier, that a refill is needed. A confirmation request may additionally be generated to the user or another designated party on behalf of the pharmacy or other product vendor.

In another aspect, the present invention provides methods for monitoring adherence with a treatment protocol. In some embodiments the method comprises monitoring a characteristic of a product related to a treatment protocol. In some embodiments, the product may comprise a single product or plurality of products including, but not limited to a pharmaceutical product, vitamin, nutrition aid and/or the like. In some embodiments, the characteristic may comprise amount, quantity, mass, weight, volume, capacitance, opacity, light absorption or reflection, or similar physical and/or chemical characteristic. In an embodiment, the monitoring of the characteristic may comprise determining a change in the characteristic.

Embodiments of the methods of the present invention may further comprise: communicating the change in the characteristic to a person practicing the method and/or a third party, e.g. a health care provider, product supplier, pharmacy, pharmacist, clinical trials provider, insurer, relative, friend or the like. Embodiments of the methods of the present invention may still further comprise capturing data relating to the change in the characteristic and storing and/or communicating that data to the person practicing the method and/or a third party. Embodiments of the methods of the present invention may further comprise alerting a person (i.e., a user) being monitored of a need to take or use the product and/or alerting the third party of the need to take or use the product. Additional features of embodiments of the method of the present invention will be apparent to those of ordinary skill in the art in view of the description and figures herein. An embodiment of the method of the present invention may be practiced using an embodiment of an apparatus, or a system of the present invention, or may be practiced using another apparatus and/or systems.

In a further aspect the present invention provides methods for monitoring the amount of a product. Embodiments of such methods may comprise similar steps to those in a method of the present invention for monitoring adherence to a treatment protocol. In embodiments, the method for monitoring the amount of a product may further comprise alerting the third party that the product has reached a predetermined characteristic, for example an amount. Such embodiments, for example, could alert a pharmacist, health care provider and/or other product supplier, that a refill is needed.

### BRIEF DESCRIPTION OF THE DRAWINGS

Examples of embodiments of the present invention are set forth in the attached Figures.

FIG. 1: Examples of an apparatus according to embodiments of the invention are shown in FIG. 1a. An exploded figure of one example is shown in FIG. 1b.

FIG. 2: An example of a combination of components of an apparatus according to one embodiment of the invention is shown in FIG. 2. An apparatus may comprise some or all of the components shown.

FIG. 3: A block diagram of an example of a combination of components according to one embodiment of an apparatus of the invention is shown in FIG. 3.

FIG. 4: An example of a measurement process according to one embodiment of the invention is shown in FIG. 4. Data is measured, stored, and either transmitted real-time or asynchronously depending on the device's configuration and available connections.

FIG. 5: An example of a transmission process according to one embodiment of the invention is shown in FIG. 5. Transmission of measurement data may be real-time or asynchronous depending on the device's configuration and available connection.

FIG. 6: An example of an Adherence Monitoring System according to one embodiment of the invention is described in FIG. 6.

FIG. 7: An example of a Product Refill System according to one embodiment of the invention is described in FIG. 7.

FIG. 8: FIG. 8 shows an exterior view of one embodiment of an apparatus having 5 dispensers.

FIG. 9: FIG. 9 shows an exterior view of one embodiment of an apparatus having 10 dispensers.

FIG. 10: FIG. 10 shows an exterior view of one embodiment of an apparatus adapted to an existing dispenser configuration having days-of-the-week dispensers.

FIG. 11: An apparatus example adapted to an existing dispenser configuration according to one embodiment is shown in FIG. 11, along with two components for coupling the dispenser to a load cell scale.

FIG. 12: An alternative embodiment of an apparatus adapted to the dispensers shown in FIG. 11 is shown in FIG. 12.

FIG. 13: A device process flow schematic which addresses and accounts for load cell drift according to an embodiment of the invention is shown in FIG. 13.

FIG. 14: A website process flow schematic according to an embodiment of the invention is shown in FIG. 14.

FIG. 15: An alternative embodiment of an apparatus adapted to the dispensers shown in FIG. 11 is shown in FIG. 15.

### DEFINITIONS

As used in this specification, the singular forms “a,” “an,” and “the” include plural referents unless expressly and unequivocally limited to one referent.

“Adherence,” as used herein, refers to the degree of patient compliance with care providers' recommendations, or in some cases compliance with a self-directed treatment protocol, about the daily timing, dosage, and frequency of product use. In many examples, the product is a pharmaceutical product (e.g., a medication). Nonadherence, then, is used synonymously herein with noncompliance, and represents any instance in a treatment protocol where planned or scheduled use of a product, such as a medication, is missed or skipped. In some cases it may also refer to overuse of a product (i.e., more frequent use than planned).

A “treatment protocol,” as used herein, may refer to any plan to use a product according to a particular schedule. The treatment protocol may be a prescription regimen, but it may also refer to use of a non-prescription product. The phrase “product regimen” is used interchangeably with treatment protocol herein.

“Enclosure,” as used herein, refers to a vessel, container, or dispenser for holding a product related to a treatment protocol.

A “load cell,” as used herein, refers to a type of transducer that creates an electrical signal based on the force (e.g., weight) being measured and is a component of many types of scales. A load cell may be used to detect the weight of products based on the strength of the electrical signal generated by the force of the weight. A strain gauge is a type of load cell, and the term “strain gauge” is used interchangeably with load cell herein.

“Load cell drift” or “load cell creep” (or simply “drift” or “creep”), as used herein, refers to the tendency of a load cell that remains on at all times to exhibit inaccuracies due to a variety of factors such as metal fatigue, variations in the supplied electrical current, changes in the ambient temperature, and other physical parameters.

### DETAILED DESCRIPTION

Monitoring adherence with product regimens, such as prescription medication regimens, may substantially enhance correct evaluation of the effectiveness of the product or prescription, encourage better adherence, and more efficiently generate prescription refill requests.

Product dispensers may include containers or enclosures used to hold the product, such as medicine that may be in liquid, solid, or aerosol form. Ointments or creams may also be enclosed in their usual dispenser, e.g., a crimp tube. Product dispensers will generally have at least one container but may have more. Many individuals who take a variety of medicines use a dispenser with a container for each day or even multiple containers per day, e.g., Monday through Friday with AM/PM containers. Medicine dispensers are typically made of plastic and are simply containers for said medicine, or when considering the daily form, dispensers may act as passive reminders for patients to take the medication.

Patients, physicians, patient family members, other health care providers, insurers, pharmacists, government regulatory agencies, and/or drug companies may be among the groups of stakeholders that could benefit greatly from a smarter form of this dispenser. Drug companies conducting human clinical trials typically rely on pill counts to determine how many pills a patient took between visits. Physicians must rely on a patient's word when determining the efficacy of treatment. Patients who are less than forthcoming may put themselves in danger by misleading physicians about their adherence to or compliance with a medicine regimen. In extreme circumstances, patients' lives may be in danger if this misleading information leads to inappropriately increased dosages.

Additionally, pharmacies could benefit from a smarter dispenser, specifically with more information regarding medication usage. Current pharmacy resource management systems may not accurately forecast demand for a certain drug and may result in pharmacies under/over-ordering those drugs based on historical demand. For generic drugs, poor forecasting may be a less pressing issue; but with an increasing number of medicines costing hundreds or even thousands of dollars per dose, poor forecasting could significantly impact a pharmacy's ability to operate. A smarter medicine dispenser would assist in measuring usage more accurately, thus allowing pharmacies to better predict upcoming demand and streamline their operations.

Memory and understanding are significant factors contributing to individuals' nonadherence. A patient may not understand a new medicine regimen due to its complexity; or he may forget, due to new dosing times and the changes in behavior that are required (e.g., remembering to take a new medication after every meal). An effective adherence monitoring/preemptive intervention system must take these factors into account.

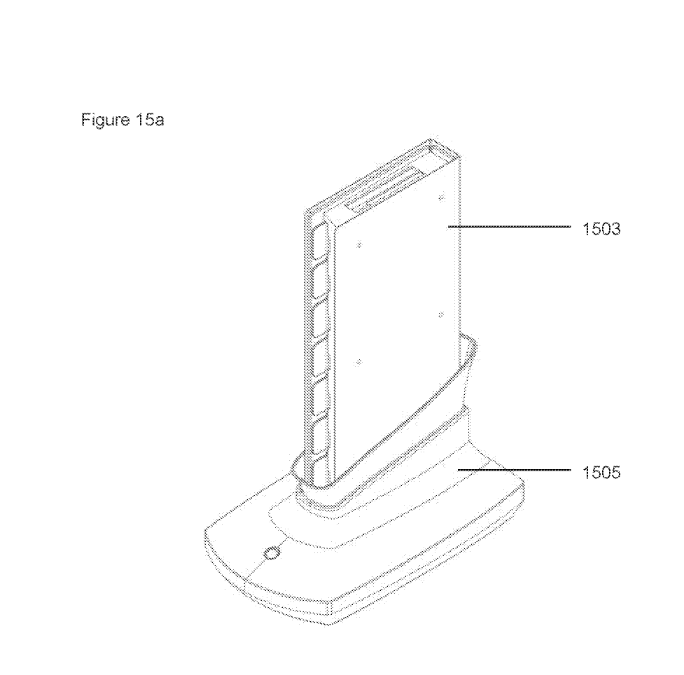

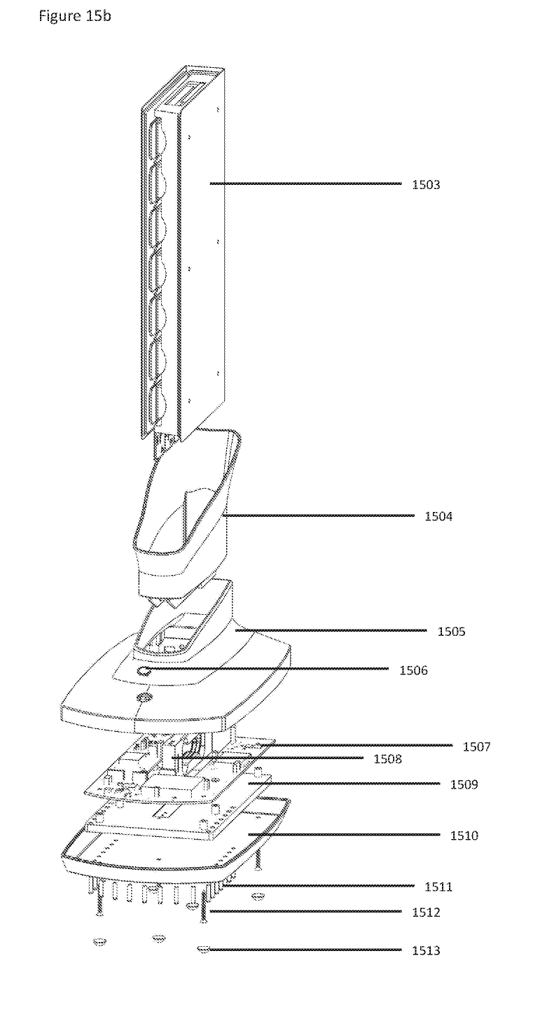

The systems described herein are designed to require little to no behavioral change from the patient. Rather than introducing additional actions a patient must remember, this system requires little to no input from patients through the apparatus. Apparatuses are designed for intuitive use based on how a patient currently takes his medication. As a non-limiting example, FIG. 15 shows an apparatus that accepts a medicine box, commonly used throughout assisted living facilities. For a patient to use this apparatus, he must simply store his medicine box in the receiver rather than his counter-top so that configuration, management, and interventions may be handled remotely, without any patient effort.

In an embodiment, an apparatus for monitoring adherence to a treatment protocol comprises an enclosure for holding a product, such as a pharmaceutical product, and a measuring apparatus or other measurement means for determining a characteristic of the product within the enclosure. The measurement means may be capable of determining the amount, quantity, mass, weight, volume, capacitance, opacity, light absorption or reflection, or similar physical and/or chemical characteristic of the product in the enclosure and also capable of detecting an incremental change in that characteristic.

In some embodiments, the characteristic is weight and the measurement means is a scale. The scale may measure the weight of the contents of the enclosure and may include one or more load cells. The scale may be sensitive and accurate enough to detect an incremental weight difference, such as a single dose difference, e.g., in decigrams, centigrams, milligrams and/or micrograms.

The apparatus may be programmed to measure weight of the remaining medication at regular intervals or at specified times of day, or triggered by specified events such as removal of a medicine container. The apparatus as a computing device may include a processor, memory, sensors, timer, power source, RFID antenna, switches, and/or GPS transceiver. It may also include a user interface and display and a network interface. In some embodiments, the apparatus comprises at least one physical or wireless connection with the internet or other networking systems.

Another aspect of the invention is a system for monitoring adherence to a treatment protocol for taking medications, including an apparatus as described above, a component for transmitting data, a component for receiving data, a component for processing the data as compared to previously received data or a preset baseline, and a component for transmitting an alert to a designated party in case of nonadherence. The system may also include a component for transmitting feedback to the apparatus. For example, a determination of adherence to a treatment protocol may simply generate feedback to confirm adherence.

In some embodiments, the system detects a change or lack of change in a measured characteristic of a product (e.g., a pharmaceutical product) enclosed in the apparatus. When the system detects a lack of change in weight or volume over a specified time period, it may generate an alert of nonadherence to a designated party. In some embodiments the detection of an expected (preset) change in weight or volume generates feedback to the apparatus indicating adherence.

Other aspects of the invention include a system for monitoring need for a prescription refill, including an apparatus as described above, a component for transmitting data, a component for receiving data, a component for processing the data as compared to previously received data and/or a preset baseline, and a component for transmitting an alert to a designated party if the data processing indicates that a prescription refill is needed.

In some embodiments, detection of a weight or volume below a preset baseline weight or volume triggers transmission of a prescription refill order request to a pharmacy, medical distributor, or other designated party. Optionally, the system may include a component for sending a confirmation request to a designated party.

Another aspect of the invention is a method for monitoring patient adherence to a treatment protocol for taking medications, including providing a server or a data receiving application, receiving measurement data from an apparatus as described above, processing measurement data against previously received data or a preset baseline, and generating an alert to a designated party if the processing yields a specified result. In some embodiments, when the processing leads to a specified result of determination of nonadherence with a prescription regimen, an alert may be sent to a designated party who may be the patient, a clinical trial entity, a healthcare provider, a caregiver, a pharmacy, a family member of the patient, or some combination of these. In some embodiments, the specified result is a determination that there is a need for a prescription refill. In some embodiments, the method includes sending an alert to a designated party that a prescription refill is needed.

The apparatus and systems of the invention may be customized to meet the needs of particular circumstances for each individual in need of adherence monitoring.

### Apparatus

In an embodiment, an apparatus of the present invention comprises a vessel or enclosure for holding a product and a measuring apparatus for determining the amount of product within the vessel or enclosure. The product may be a consumable product. The product may be a pharmaceutical product and may take any form, including, but not limited to, a tablet, powder, a capsule, a gel cap, a liquid, a liquid suspension and/or mixtures thereof. The enclosure may comprise a dispenser, a vessel or the like, and may include a cap or other means for retaining the product.

In embodiments, the means for determining the amount of a product (e.g., a pharmaceutical product) within the enclosure may comprise a measurement of a physical attribute of the product, including but not limited to weight, volume, mass, capacitance, opacity and the like. In embodiments, the apparatus may comprise a scale, optical measurement apparatus, means for measuring volume and/or similar measurement apparatus.

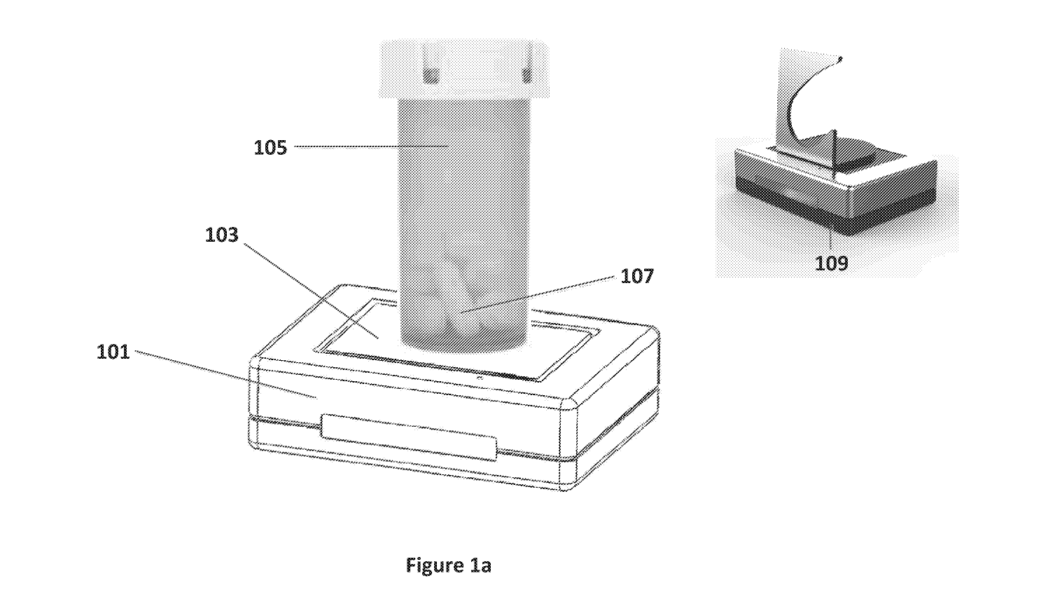

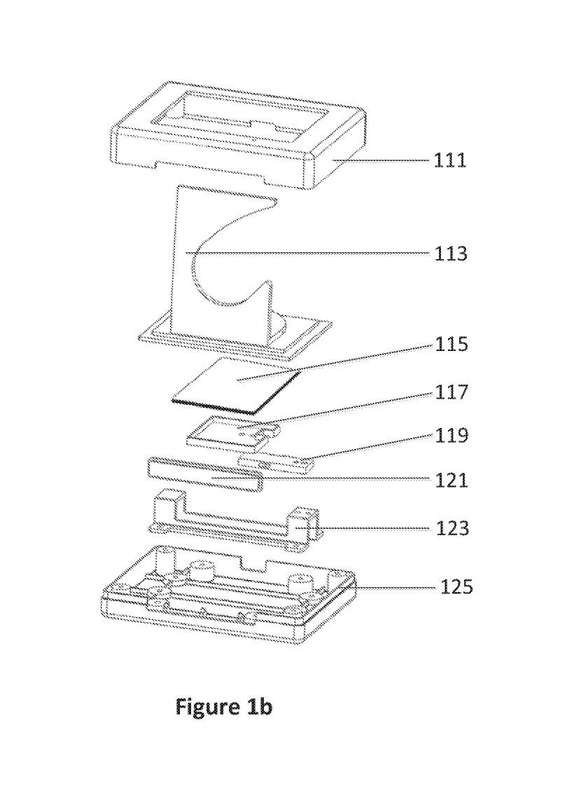

In an embodiment, an apparatus of the present invention may comprise a receiver (e.g., tray with raised edges) adapted to receive an enclosure for holding a pharmaceutical product in combination with a measurement apparatus wherein the measurement apparatus provides means for determining a physical attribute of the pharmaceutical product; for example, the weight of product contained in an enclosure may be determined by a sensitive and accurate scale as an indicator of the amount of product remaining inside. FIGS. 1a and 1b illustrate an example of apparatus embodiments.

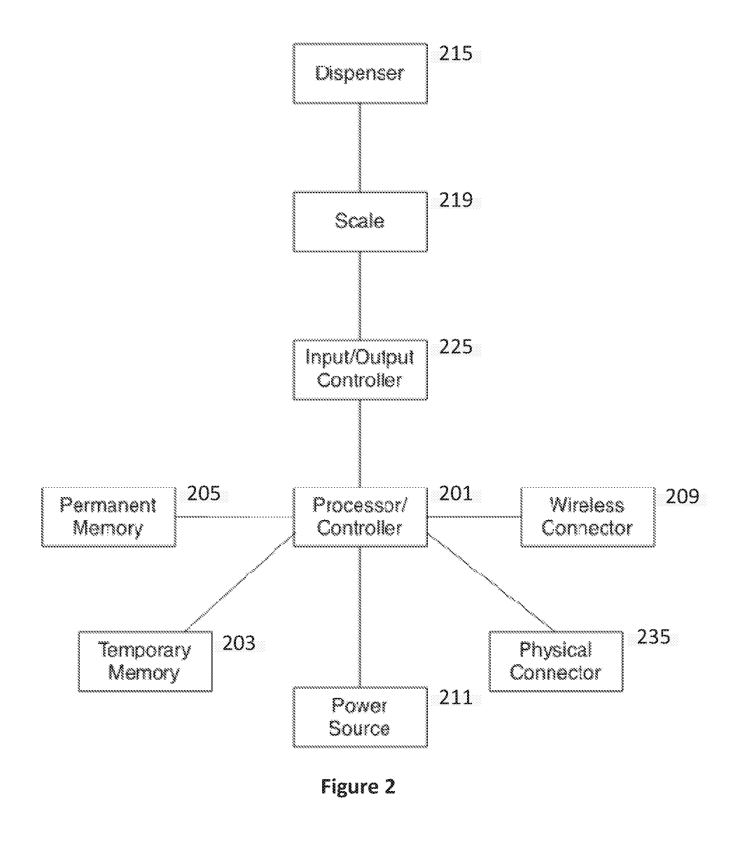

The apparatus, as a computing device, may include one or more processors that are communicatively coupled to a memory. FIG. 2 illustrates a processor 201 communicatively coupled to temporary memory 203 and permanent memory 205. Thus the apparatus may store measurement data for transmission at a later time (i.e., asynchronous transmission). A bus may communicatively couple one or more components of the computing device. One or more processors can execute computer-executable program instructions and/or access information stored in the memory and may include any number of processing devices. One or more processors 201 may include or may be communicatively coupled with a computer-readable medium such as a memory that stores instructions. The processor(s) may execute the instructions stored in the memory and thereby perform one or more operations described herein. The one or more processors 201 may comprise a microprocessor, a digital signal processor (DSP), an application-specific integrated circuit (ASIC), one or more field programmable gate arrays (FPGAs), or state machines. The processor may further comprise a programmable electronic device such as a PLC, a programmable interrupt controller (PIC), a programmable logic device (PLD), or other similar devices.

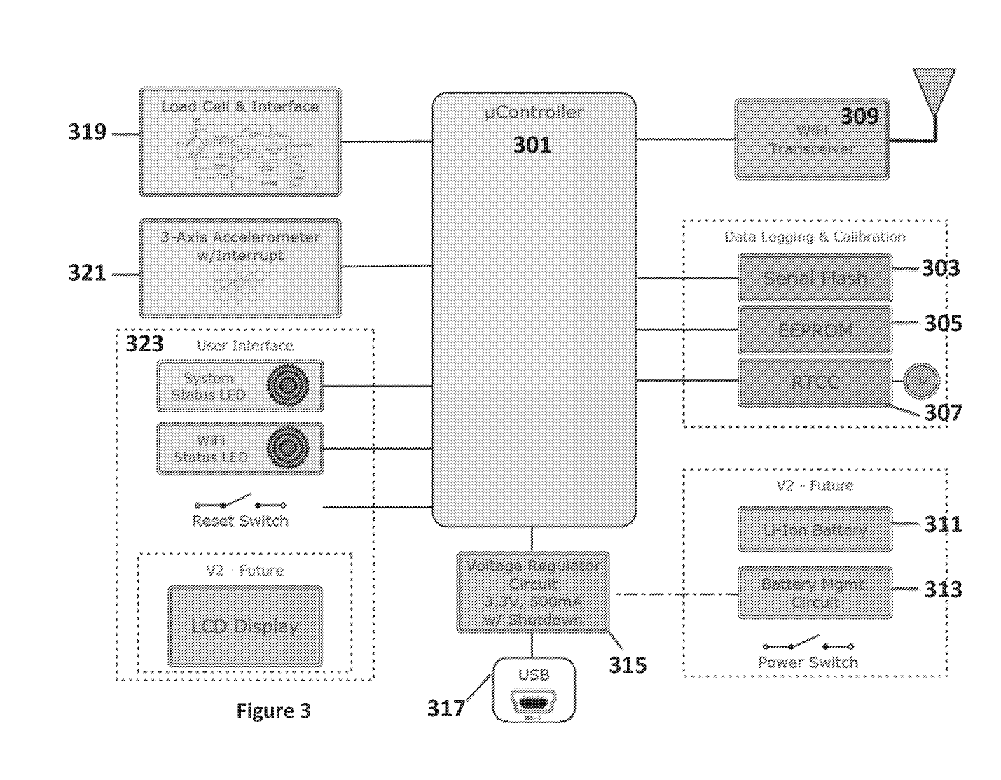

FIG. 3 is a block diagram of one embodiment of the present invention showing examples of components that may be used to implement an apparatus of the present invention. In embodiments, an apparatus of the present invention comprises one or more processors, such as microcontroller 301 shown in FIG. 3. The one or more processors execute computer-executable program instructions stored in a memory.

In embodiments, an apparatus of the present invention may further comprise memory. FIG. 2 shows temporary memory 203 and permanent memory 205. The memory may include any suitable computer-readable medium. The memory may be digital or analog and may be temporary (e.g. flash) or substantially permanent. The computer-readable medium may include any electronic, optical, magnetic, or other storage device capable of providing a processor with computer-readable instructions or other program code. In one embodiment an apparatus of the present invention comprises serial flash memory 303 and an EEPROM 305 as shown in FIG. 3. Non-limiting examples of a computer-readable medium include a floppy disk, CD-ROM, DVD, magnetic disk, memory chip, ROM, RAM, an ASIC, a configured processor, optical storage, magnetic tape or other magnetic storage, or any other medium from which a processor can read instructions. The instructions may include processor-specific instructions generated by a compiler and/or an interpreter from code written in any suitable computer-programming language, including, for example, C, C++, C#, Visual Basic, Java, Python, Perl, JavaScript, and ActionScript.

The apparatus, as a computing device, may also include a number of external or internal devices such as input or output devices (e.g., a touchpad, touchscreen, display, mouse, and/or keyboard). For example, the computing device may comprise one or more input/output (“I/O”) interfaces that can receive input from input devices (e.g., scale 219 in FIG. 2) or provide output to output devices. For example, in one embodiment an apparatus of the present invention comprises one or more load cells. A load cell and load cell interface 319 may be included for measuring physical characteristics, such as weight, of products, as shown in FIG. 3. An apparatus may also receive input from various sensors. In some embodiments, an apparatus of the present invention may comprise an accelerometer to detect the position of the device or apparatus. For example, a 3-axis accelerometer 321 is shown in FIG. 3.

The apparatus, as a computing device, can execute program code. The program code may reside on any suitable computer-readable medium and may be executed on any suitable processing device. The program code may reside in the memory of the computing device. The program code stored in the memory can configure the processor to perform the operations described herein, such as evaluating current physical characteristics (e.g., weight) of a pharmaceutical product in an enclosure as compared to previously recorded characteristics of the same enclosure, and evaluating compliance/adherence to a prescription regimen.

The apparatus may also include a network interface. One or more network interfaces may include any device or group of devices suitable for establishing a wired or wireless data connection to one or more data networks. Non-limiting examples of a network interface include an Ethernet network adapter, a modem, and/or the like. FIG. 2 shows a wireless connector 209 and a physical connector 235. The network interface can transmit signals that are modulated with or otherwise include data generated by one or more applications executed by the processor. The network interface can also receive signals that are modulated with or otherwise include data that may be used by one or more applications executed by the processor.

In embodiments, an apparatus of the present invention may further comprise a transceiver. The transceiver may be configured to transmit and/or receive data via a network, including but not limited to a wireless internet network, a cellular data network, a communications network, or the like. For example, in one embodiment an apparatus of the present comprises a Wi-Fi transceiver 309 as shown in FIG. 3. Data may be transmitted by Bluetooth, Ant+, TCIP and/or similar communication protocols.

In embodiments, an apparatus of the present invention may further comprise a timer. The timer may be an analog or digital clock, with, or without, a display. For example, in one embodiment an apparatus of the present invention comprises a real time clock and calendar device (RTCC) 307 as shown in FIG. 3.

In embodiments, an apparatus of the present invention may further comprise a global positioning satellite (GPS) transceiver. The GPS transceiver is capable of receiving signals from a GPS satellite providing location data.

In embodiments, an apparatus of the present invention comprises a power source. Power source 211 is shown in FIG. 2. In some embodiments, the power source is a battery. Optionally the battery is a rechargeable battery. The battery may be any type battery known to one having ordinary skill in the art. For example, the embodiment shown in FIG. 3 comprises a Li-Ion battery 311. In one embodiment, an apparatus of the present invention further comprises battery management logic and/or circuitry 313 for power management and controlling recharging of the battery 311. In one embodiment, an apparatus of the present invention comprises a USB port 317 for receiving power and voltage regulator circuitry 315 for regulating the incoming voltage.

In embodiments, an apparatus of the present invention comprises a user interface comprising one or more indicators (e.g. light- or sound-emitting devices), user input mechanisms (e.g. buttons, switches, touchpad, touchscreen), and/or displays (e.g. LCD display). For example, in one embodiment shown in FIG. 3 a user interface 323 comprises an LED indicating a status of the apparatus (e.g. on/off, error conditions, time to take medication reminder, etc.), an LED indicating whether the apparatus is connected to Wi-Fi, a reset switch for resetting the apparatus back to an initial state, and an LCD display for displaying information to the user.

The apparatus may further include a more sophisticated user interface for entering details about prescription regimens, prompts for measuring weight of a single dose, etc., or existing computing devices (e.g., smart phone, laptop, etc.) may be used to enter inputs to the apparatus and/or receive outputs from the apparatus. Computer readable media may be incorporated into the apparatus or may facilitate the use of existing computing devices (e.g., via software or a smart phone application) to interface with the apparatus.

In embodiments, an apparatus of the present invention further comprises connections between and among the receiver, measurement means, memory, transceiver, timer, and/or GPS transceiver. In embodiments, one or more of these features may be implemented in an integrated circuit.

By way of non-limiting example, in an embodiment an apparatus of the present invention shown in FIG. 3 comprises a medicine dispenser that weighs its contents via load cell and interface 319 at regular intervals and tracks that information via temporary or permanent digital memory. This weight data (or data regarding another characteristic such as capacitance) may be transmitted real-time to the internet for consumption by an appropriate web service. Data may also be stored for asynchronous transmission of the most recent weight via the Internet or a direct connection to a computer, including but not limited to wireless connections, e.g. Bluetooth, and physical cables, e.g. USB. In one embodiment, the data is transmitted over a USB connection through USB port 317.

In some embodiments, an apparatus of the invention may be adapted to receive existing dispensers (e.g., a dispenser already familiar to a user). In an embodiment the dispenser has at least one container for medicine but may have more, e.g., a dispenser with a container for each day of the week. Various dispenser configurations and various schedules for detection of weight (or other characteristics) are possible. The intervals at which the weight measurements take place may be regular based on the time of day or the time it last ran, or may be regular depending on an action taking place on the dispenser, e.g., each time it is removed from the apparatus and replaced.

An apparatus may be selected and tailored to accommodate the needs and/or preferences of an individual patient. FIG. 1a illustrates a simple apparatus example with a receiver adapted to receive a single medication enclosure or dispenser. The receiver may be advantageously adapted to receive standard sizes of prescription medicine enclosures, such as those depicted in the figures. Other product enclosures may also be compatible with this receiver example, e.g., liquid medicine bottle or ointment crimp tube (e.g., standing on a broad-based cap).

The receiver is physically coupled with a measuring apparatus component for determining a characteristic (e.g., a physical property such as weight) of the product within the enclosure. The component may be a measurement means capable of determining the amount, quantity, mass, weight, volume, capacitance, opacity, light absorption or reflection, or similar physical and/or chemical characteristic of the product in the enclosure and also capable of detecting a change in that characteristic (e.g., change of a single dose). In some embodiments the physical property is the weight of the enclosure and any medicine contained therein. Thus as shown in FIGS. 1a and 1b, e.g., a scale is provided for measuring weight of the enclosed product.

The apparatus may further comprise any variety of communication means. The apparatus may optionally generate a signal for notifying a user of an event (e.g., the need to take medicine). For example, a green light may indicate medication needs to be taken, while a red light may indicate medication has already been removed from the dispenser and is not due to be taken again until the next day (or some other future time). Some users may prefer sound cues instead of or in addition to light indicators. An apparatus may additionally be programmed to sound an alert to the patient that it is time to take medications at a certain time, if the medication has not already been removed from the dispenser, e.g., by sounding an alarm similar to an alarm clock, or sounding a verbal voice instruction that it is time to take medications. A display may additionally indicate the number of doses of medication remaining inside. The apparatus may further comprise a timer and/or any of the other features disclosed herein, singularly or in combination. Thus, in some circumstances, a patient or user may be able to monitor his or her own adherence to a treatment protocol with the assistance of an apparatus of the invention, with or without the additional assistance of the systems described below.

FIGS. 1a and 1b illustrate an exterior view and an exploded view of an apparatus of one embodiment of the present invention. In particular, FIGS. 1a and 1b illustrate an apparatus 101 for implementing the methods and systems described herein. Apparatus 101 comprises a receptacle or receiver 103 configured to receive a vessel, dispenser, or enclosure 105 for holding medicine, such as a typical prescription pill bottle. The medication or other product 107 is contained in the enclosure.

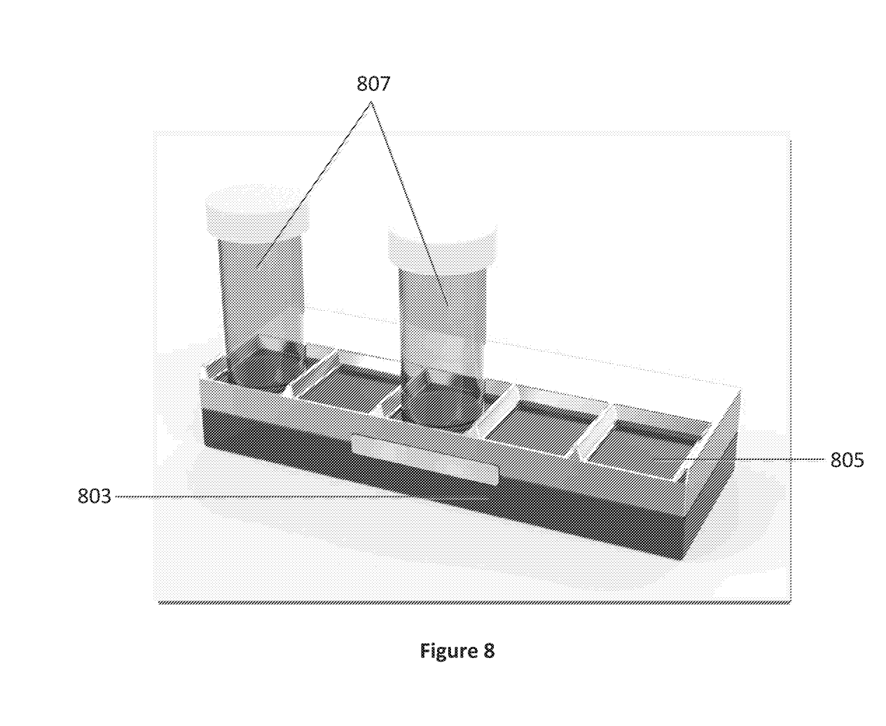

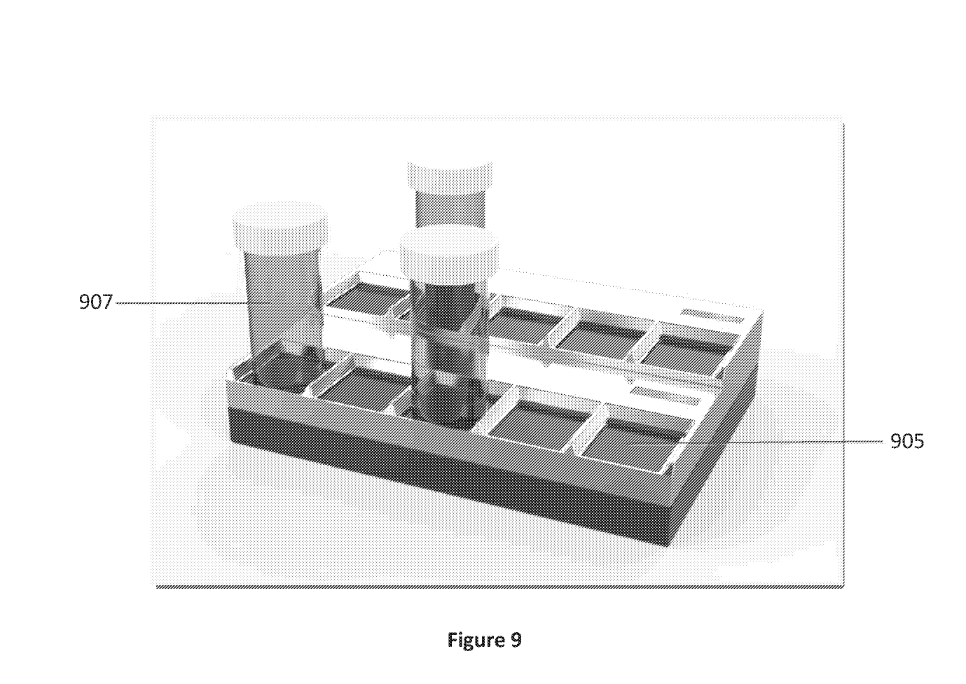

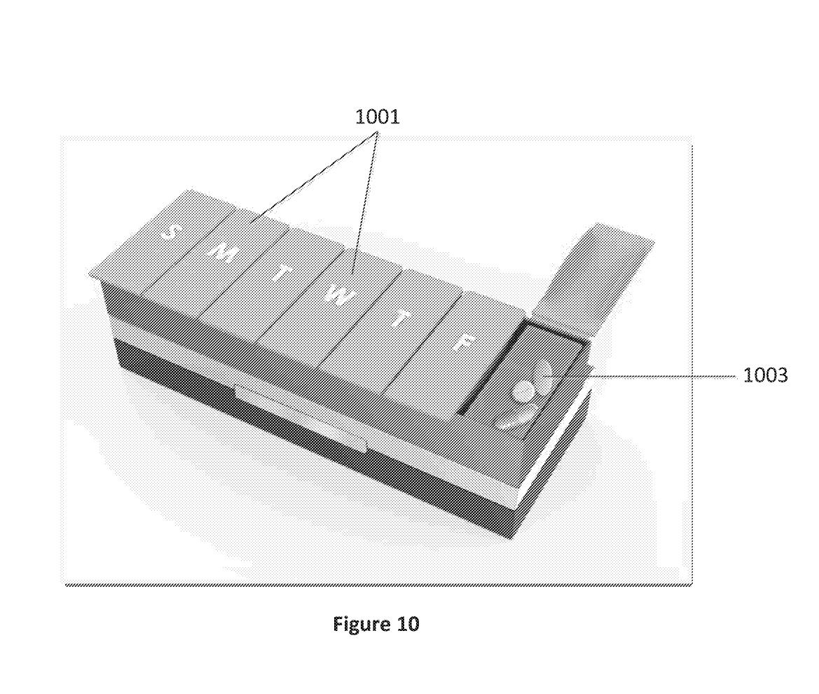

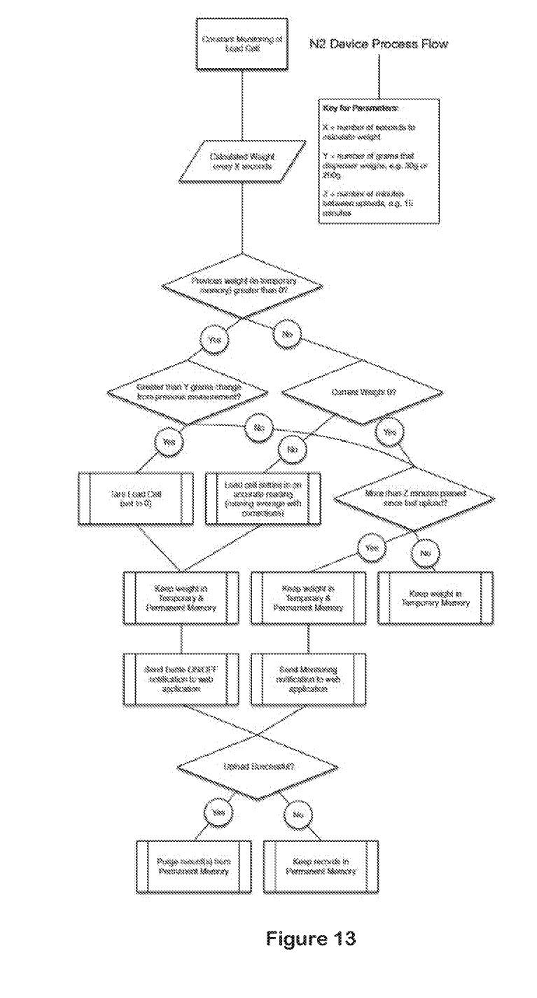

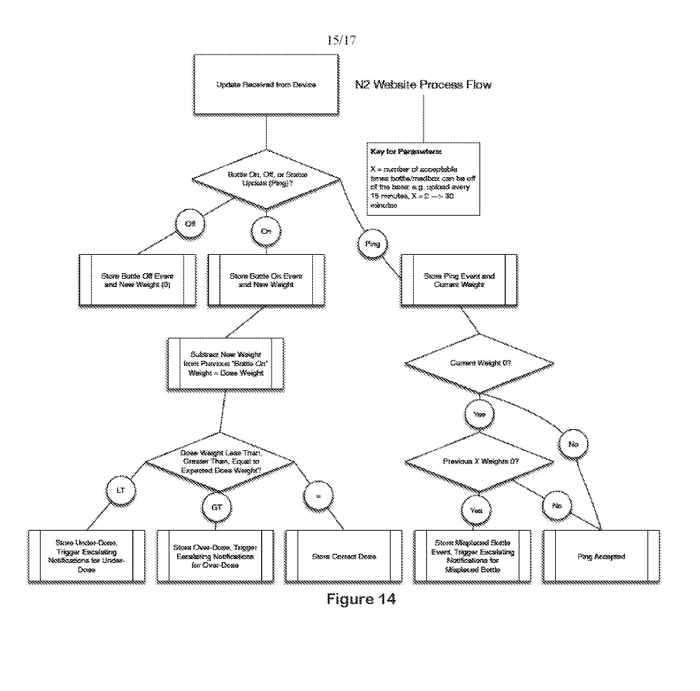

In another dispenser configuration shown in FIG. 8, a patient who takes 5 medications daily may benefit from using an apparatus that allows each of the 5 medications to be monitored separately. Some embodiments of an apparatus may be modular. The apparatus of FIG. 9 utilizes two modular units (as in FIG. 8) configured to include 10 receivers for 10 different medication enclosures. This example may be suitable for a patient who takes a number of different medications on various schedules. Alternatively, a patient who takes 5 medications daily, all at the same time, may achieve better adherence or compliance with an apparatus as shown in FIGS. 10-12, where a variety of different medications, all to be taken at the same time, are contained in the same enclosure. A patient who takes a variety of medications at different times each day can benefit from the devices shown in FIGS. 13 and 15. The analysis and output may also be tailored to accommodate particular circumstances, based on user inputs related to prescription regimens. A user interface and display may facilitate user input, or other means may be used to communicate with the apparatus (e.g., smart phone with designated application, desktop or laptop computer, etc.).

### Systems and Methods

The present invention also provides methods and systems for monitoring adherence to a treatment protocol. The methods and systems may advantageously utilize an embodiment of an apparatus of the present invention, however are not so limited and may use other apparatus and systems.

Methods and/or systems of the present invention may be advantageously implemented using an apparatus of the present invention, such as the apparatus depicted in FIGS. 1a and 1b, an apparatus with dispensers as illustrated in FIGS. 8-12, or other apparatus configurations.

In an embodiment of the present invention, the change in a physical characteristic of a product in a vessel, related to use of the product, is used to monitor adherence. A physical characteristic may include weight, mass, volume, capacitance, opacity or other characteristics of a product, e.g., a medicine.

In an embodiment, an adherence or compliance monitoring system of the present invention uses the weight of medication or other product consumed at regular intervals. As a user takes incremental portions of the product out of the vessel or other enclosure, the weight of the remaining product will correspondingly decrease. In an embodiment the incremental portions are medication doses, and adherence is monitored for a particular prescription regimen. In such an embodiment, weight reduction is used to monitor the levels of medication that have been taken by the patient and to measure/determine adherence to a medicinal regimen.

Standard weights for individual pills/doses are recorded, including tolerances if necessary. A patient's medicinal regimen is also recorded (e.g., via website), including specific medications, dosage size, and dosage frequency. Depending on the complexity of the medicinal regimen, the apparatus and input settings may be tailored to the needs of an individual patient. For example, FIGS. 8-9 demonstrate apparatus configurations that may allow separate scheduling and adherence monitoring for each different medication. Alternatively, FIGS. 10-12 show embodiments that may be better suited to a patient who takes several medications all at the same time each day.

The weight of a patient's remaining medication may be measured at regular intervals, in some embodiments. This weight information is either stored for later use or transmitted real-time (i.e., as soon as a measurement is taken) for evaluation and interpretation of the results. Necessary parties, e.g. primary caregivers, pharmacists, physicians, or pharmaceutical companies, may access this information. Additionally, specific parties may be alerted automatically (e.g., via text or e-mail message or phone call) in certain circumstances (e.g., no medication has been removed for some preset period of time). Hierarchical alerts may be customized per patient, per medication, and per adherence condition (e.g., missed dose, taken too much, taken too little). The exact amount of weight reduction used in conjunction with the weight of one pill/dose of said medication allows evaluators (human or electronic) to determine the amount removed since the previous measurement. The involved parties (or technologies) evaluate all applicable data and draw conclusions about patient adherence to the medicinal regimen.

In some embodiments, a different characteristic may be measured, such as capacitance. For example, packaging (e.g., blister pack) may be designed to facilitate measurement of capacitance of the packaging including medications, and measured capacitance reflects a change in the contents of the packaging. Thus removal of medication from the packaging results in a change in the capacitance. In one embodiment, a capacitance measuring device is a clamp.

### Addressing Creep (or Drift) Exhibited by a Load Cell

In some embodiments, computer readable media for use with the device and/or the device itself may be designed to address the tendency of a load cell to exhibit “creep” or “drift” over time. Electrical based load cells, such as strain gauges or piezoelectric load cells, determine strain/force/weight through changes in electrical resistance or output within the mechanism. When these load cells run constantly, they are susceptible to inaccuracies due to a variety of factors: metal fatigue, variations in the supplied electrical current, changes in the ambient temperature, among others. Each of these factors can affect the way in which the electricity runs through the load cell, thus resulting in inaccurate measurements. These inaccuracies are typically referred to as “creep” or “drift” and measured in a percentage of the maximum capacity of the load cell over a specific period of time.

In precise consumer load cell applications such as a jeweler's scale, creep does not present an issue because the scale is designed with an On/Off button and perhaps a Tare button. When a load cell is first turned on and/or receives a tare, software within the device effectively “zeroes out” the sensor's results with a new baseline from which the measurements will be taken. This new baseline means that any factors that contribute to creep are irrelevant and the results will be accurate.

In a device designed to track medication usage by weight, such as the device of the invention described herein, the device must constantly run in order to capture exact dosing times. Because they are constantly running, these devices are especially susceptible to inaccuracies due to creep. While some load cells may only creep a fraction of a gram over days and weeks, this creep could exceed the weight of medication dosages, which would render the device useless.

Additionally, these devices should not have a Tare button (or at least should not be easily accessible) because the reliability of the device would come into question. These devices are designed to track medication usage on their own and if they are reliant on user input for accurate measurements, the device is no better than current alternatives such as questionnaires.

Device software can be written to compensate for creep, but since each load cell will creep differently (and differently over time), the reliability, feasibility, and scalability of successful software based compensation is doubtful at best. A simple and flexible solution would eliminate the problem of creep in devices that must constantly run.

The creep that can occur between dosing times may exceed the actual weight of the dose, which could as small of a measurement as several centigrams. However that same creep would not exceed a larger percentage of the maximum weight of the load cell, e.g. 10% of a 400 g load cell→40 g. This larger percentage may be referred to as the threshold. The threshold may be determined based upon the quality of the load cell, temperature shielding, etc. and may be used in the design of the device and medicine container.

The design of the device may beneficially require the user to remove the container holding the medication from the load cell (and mounting plate) in order to take the medication. For example, if a medicine box is being measured, walls may be designed around the medicine box that would prevent access to the medication without removal of the unit. Thus the user must remove the medicine box from the device in order to access the medication. Similarly, a medicine bottle must be removed from the device in order to be opened.

The empty weight of the item being removed (medicine container) may be designed (or designated) to exceed the threshold, e.g., greater than 40 g in the example above. The constantly running load cell in the example device may then be able to determine a change in weight greater than 40 g, regardless of creep (over an amount of time that would far exceed any reasonable time between dosing periods).

The software within the device may be developed to constantly monitor the results from the load cell; if a weight exceeding the threshold (40 g in the example) is removed, an automatic tare is triggered. This tare may correct for any creep that occurred since the last dosing time. When the medicine container is placed back onto the load cell (and mounting plate), the software may determine an accurate weight without any consideration for creep.

To add additional reliability through redundancy, additional switches or sensors may be added to the device to detect the removal and replacement of the medication container. When used in conjunction with threshold detection, these additional switches and/or sensors may be used to verify that an expected action occurred. For example, a mechanical switch may be used to detect the presence or absence of the container in the enclosure, as the mechanical switch is depressed when the container is in place and the switch is released when the container is removed. Alternatively, an optical sensor, such as an optical gate, may be used to detect the presence or absence of the container in the enclosure. Similarly, sensors, such as proximity sensors, may be employed to detect the presence or absence of the container in the enclosure of the container and to provide redundancy.

The N2 device process flow diagram in FIG. 13 shows the process a device may follow when connected to a web application designed to monitor medication adherence.

Thus the devices, methods, and systems of the invention may obtain accurate load cell measurements of medication weight, despite being in an “always on” state, through a combination of design and event-driven automatic tare.

The N2 website process flow diagram in FIG. 14 shows the process a web application may follow after it receives an update from a device as described in FIG. 13.

### Data

Conclusions regarding patient adherence will be used in a variety of considerations relating to the healthcare field, including, but not limited to, drug trials, appropriate dosage size, treatment efficacy, drug interaction effects, drug abuse, and historical compliance.

In some embodiments, systems of the invention may utilize aggregate data (e.g., for individual users, some class of users, or all users of such systems) to identify trends or patterns in adherence or compliance. In some embodiments, such data may enable preemptive intervention, such as an additional instructions or alerts to take a medication before a period of time when the user often misses his or her dose of medication.

In other embodiments, systems of the invention may allow a care provider (e.g., prescription writer) to predict likelihood that a particular patient will adhere to a treatment protocol. For example, if a patient has a good track record for single daily dose medications, taken in the morning, but tends to be nonadherent to treatment protocols that require additional doses to be taken later in the day, a physician may choose an option that allows the patient to take only a single dose each day, in the morning. Again, aggregate data from both the individual patient and all patients using such systems may assist in such predictive interventions.

Another optional feature of the described invention is a medicine refill system. The refill system sends automatic refill requests to a web service that is connected to the patient's pharmacy, physician, drug distributor, or third party (or directly to any designated party) once the weight of the medicine remaining falls below a certain threshold. Optionally, a refill system may also generate a refill confirmation request alert to the patient or a party other than the patient, who may then call the designated pharmacy to confirm the refill is needed and will be picked up. This refill request allows pharmacies to better evaluate demand and usage, resulting in streamlined operations for said pharmacies. This system is also beneficial to patients who have a tendency to forget to order refills in a timely manner, leading to missed doses while they wait for the prescription to be filled.

### Detailed Description of Figures

### Apparatus

FIG. 1: FIGS. 1a and 1b are outer and inner views of an apparatus according to embodiments of the present invention. FIGS. 1a and 1b illustrate examples of an apparatus including a scale with a load cell or strain gauge and computing components housed in a base 101, as shown in FIG. 1a. A receiver 103 may hold a single medication enclosure or dispenser 105 housing a single medicine or other product 107. The receiver may be advantageously adapted to receive standard sizes of prescription medicine enclosures, such as those depicted in the figures. An inset 109 shows a receiver/holder that cradles a prescription bottle horizontally. In either example, the dispenser is removable from the assembly and may trigger the apparatus to measure weight upon its return to the assembly. FIG. 1b illustrates an exploded version of an apparatus. A cover 111 provides protection from dust and a neat appearance. A receiver/holder 113 may be designed to accommodate a typical prescription bottle cradled horizontally. Alternatively, a receiver 103 may simply hold a dispenser upright, as shown in FIG. 1a. A tray 115 serves as a receiver for holding a product dispenser (e.g., 105 shown in FIG. 1a). Below the tray is a platform 117. A load cell or strain gauge 119 is positioned just below the platform and supports the platform 117, tray 115, holder 113, and a dispenser which encloses the product. A clear plastic piece 121 provides a view of LED notification lights from the front of the apparatus. A mounting bracket 123 holds the load cell or strain gauge in position. The base 125 holds a printed circuit board (not shown) and the mounting bracket 123. The apparatus may further include a user interface (not shown), or existing computing devices (e.g., smart phone, laptop, etc.) may be used to enter inputs to the apparatus and/or receive outputs from the apparatus.

FIG. 2: An example of an apparatus may include any or all of the components shown. The apparatus, as a computing device, may include one or more processors 201. In some embodiments, the processor 201 communicates with a scale 219 via the I/O controller 225 to measure the contents of a dispenser 215 at a specified time or upon receiving some trigger. In some embodiments the scale 219 may be replaced with another means of detecting a characteristic of the product being monitored. The apparatus also includes temporary memory 203, permanent memory 205, or both. Physical connectors 235 and wireless connectors 209 are optional. The configuration of the device will depend on the requirements of the implementation, e.g. if a real-time connection is required for medication refills, the device will include a wireless connector. The wireless connector 209 could be a Wi-Fi antenna or any other form that connects to a data network. The power source 211 will be sufficient to power the device's configuration for the necessary timeframe. The power source may come in the form of a USB cable, AC, AC/DC transformer, or DC supply such as batteries.

FIG. 3: FIG. 3 is a block diagram of apparatus components according to one embodiment of the invention. This embodiment includes a microcontroller 301 for processing, which communicates through an I/O interface with various sensors, such as a load cell and interface 319 and an accelerometer 321. The microcontroller also communicates with user interface components 323, such as an LED indicating a status of the apparatus (e.g. on/off, error conditions, time to take medication reminder, etc.), an LED indicating whether the apparatus is connected to Wi-Fi, a reset switch for resetting the apparatus back to an initial state, and an LCD display for displaying information to the user. This embodiment includes a Wi-Fi transceiver 309 as a network interface. This embodiment also includes serial flash 303 and EEPROM 305 memory components, as well as a real time clock and calendar device (RTCC) 307. A Li-Ion battery 311 and a battery management circuit 313 coordinate with a voltage regulator circuit 315 and USB port 317 to provide power to the apparatus.

### Systems:

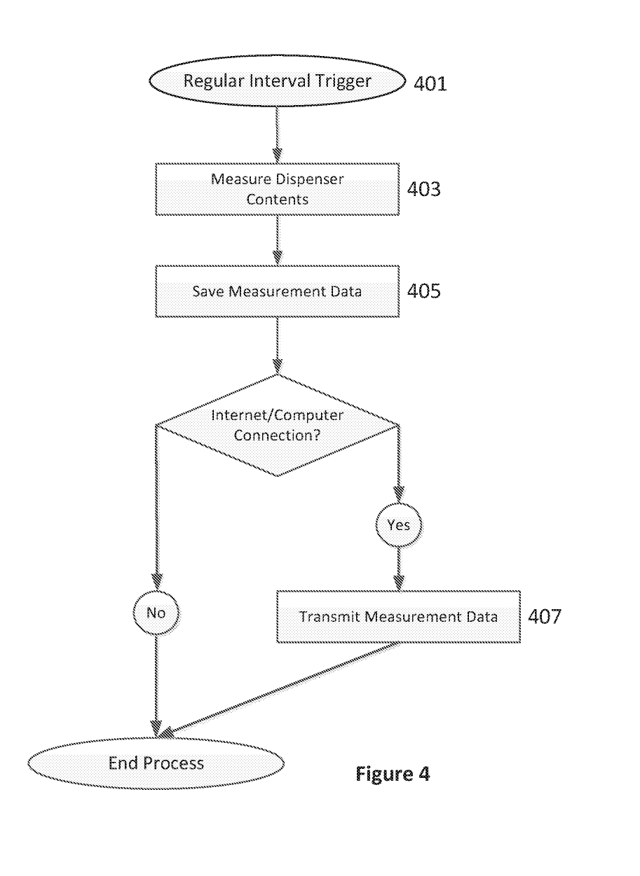

FIG. 4: An example of a measurement process according to one embodiment of the invention is shown in FIG. 4. The regular interval trigger 401 is variable as described throughout this application. The process may start due to a time of day, every X minutes, after a certain action, or due to other interval triggers. Data is generated by measuring dispenser contents 403, the data is saved 405, and either transmitted real-time or asynchronously 407 depending on the device's configuration and available connections.

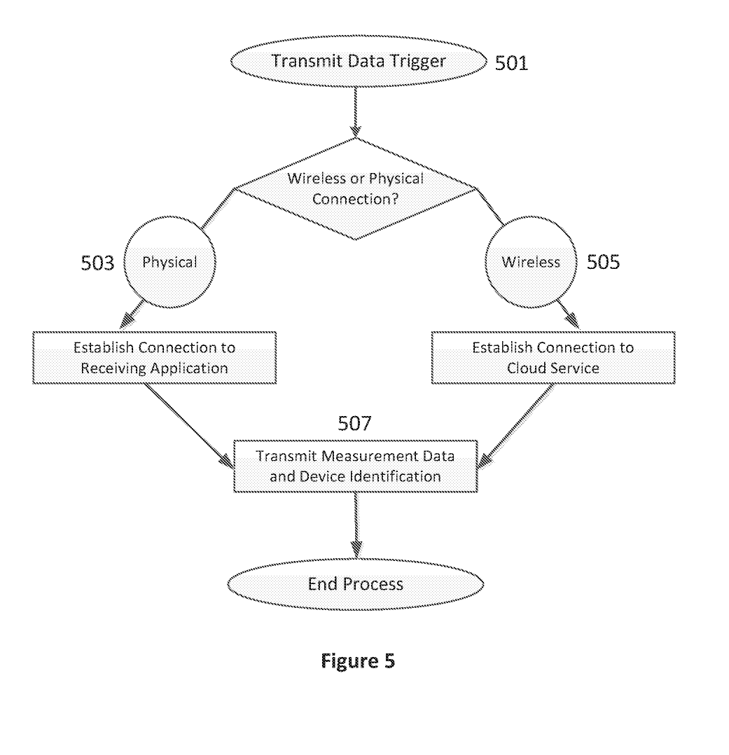

FIG. 5: An example of a transmission process according to one embodiment of the invention is shown in FIG. 5. As mentioned above, transmission of measurement data 501 may be real-time or asynchronous depending on the device's configuration and available connection. In either a physical 503 or wireless 505 connection configuration, the device requires a receiving application in the form of a web service, computer application, or other service. This receiving application receives measurement data and identifies the device 507 and may or may not send a confirmation of receipt back to the device, depending on the configuration of said application.

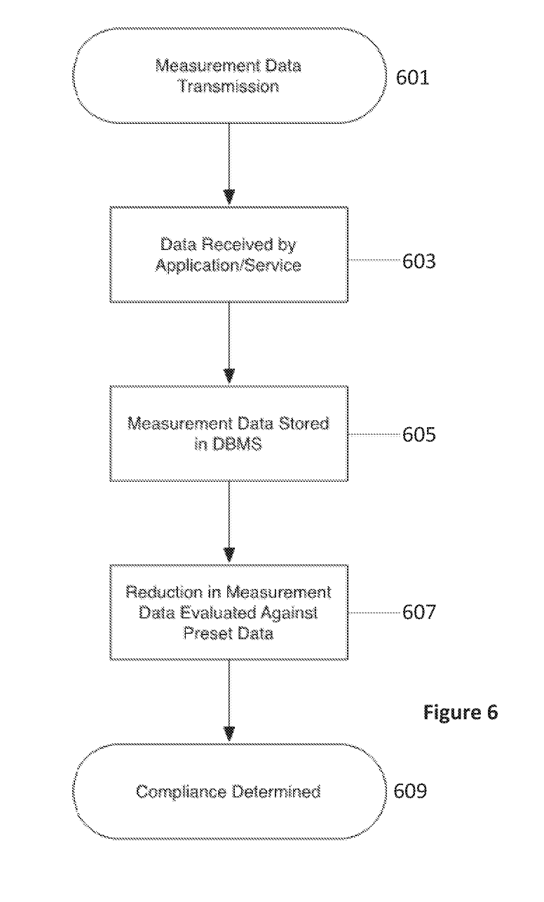

FIG. 6: An example of an Adherence Monitoring System is described in FIG. 6. Measurement data is collected as shown in FIG. 4 and then transmitted 601 to the receiving application 603, e.g., as shown in FIG. 5. This receiving application 603 evaluates the stored measurement data 605 against preset data values 607 that are dependent on the patient, medicine, regimen, and use of this apparatus and system. The evaluation determines whether or not the reductions in weight measurements are in compliance 609 with expected values (including tolerances if applicable).

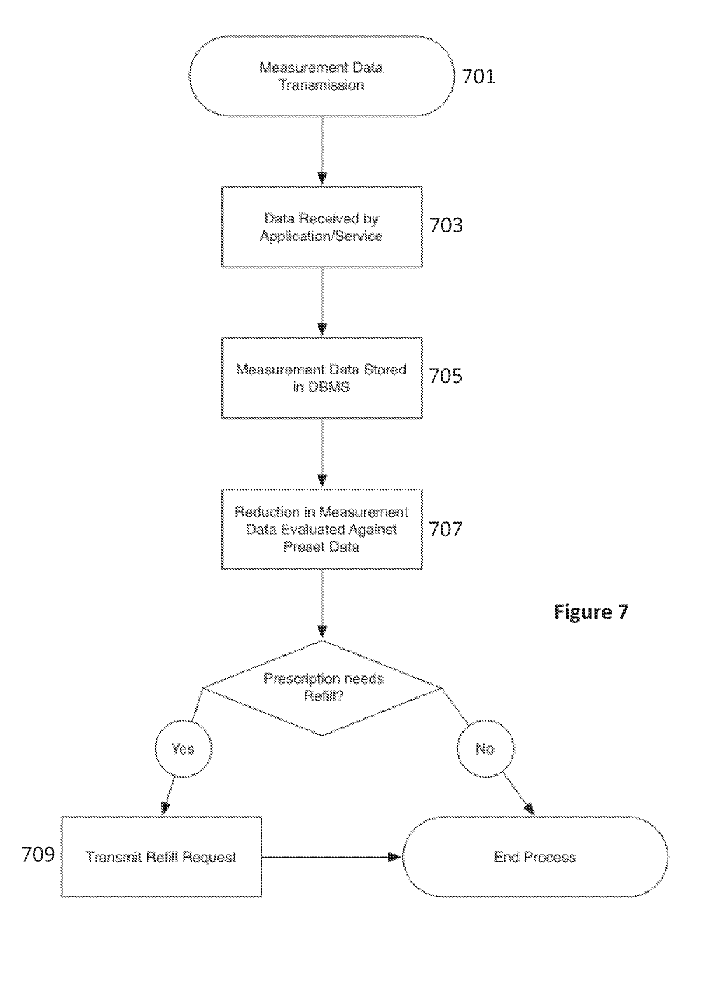

FIG. 7: An example of a Product Refill System is described in FIG. 7. Similar to the Adherence Monitoring System, the data is collected as shown in FIG. 4, transmitted 701 to the receiving application 703, e.g., as shown in FIG. 5, and then evaluated 707 by the receiving application. Rather than evaluating for adherence, this receiving application evaluates to determine whether or not the remaining medicine (i.e., stored data measurement 705) is below a preset value that will trigger a refill request. If the measurement data is below that value, the refill request 709 is sent to either a pharmacy, distribution center, or other third party that is preconfigured in the system to handle this request.

### Additional Apparatus Embodiments

FIG. 8: An apparatus according to one embodiment of the invention is shown in FIG. 8. A scale with load cell as well as computing components are housed within the base 803. This example may be appropriate for a patient who takes up to 5 medications daily, as it provides a separate receiver 805 for each of five medication enclosures 807. The weight of each enclosure may be assessed individually, such that each medication may be monitored separately for adherence to treatment protocol.

FIG. 9: An apparatus according to one embodiment of the invention is shown in FIG. 9. This example, using two modular units as shown in FIG. 8, includes ten receivers 905 for ten different medication enclosures 907. A patient who takes a variety of medications on different schedules may benefit from using such an apparatus. For example, a user interface (not shown) may be utilized to enter a separate medication schedule for each receiver slot. An indicator (e.g., colored light indicator) for each receiver slot may keep track of which medications need to be taken at any given time. For example, indicator lights for slots in the first row may be red to indicate the patient is current on those medications and is not due to take them (e.g., once daily dosage, already taken), while indicator lights for slots in the second row may be green to indicate the patient needs to take a dose of those medications (e.g., twice daily dosage, with second dose due).

FIG. 10: An apparatus according to one embodiment of the invention is shown in FIG. 10. This example includes an enclosure with seven separate compartments 1001 corresponding to days of the week. Each compartment may contain more than one type of medication or product 1003. This apparatus may be ideal for a patient who takes several medications daily, all at the same time. Particularly where a patient is familiar with this type of dispenser, this apparatus may help to achieve better adherence to a treatment protocol with minimal change to the appearance of the dispenser.

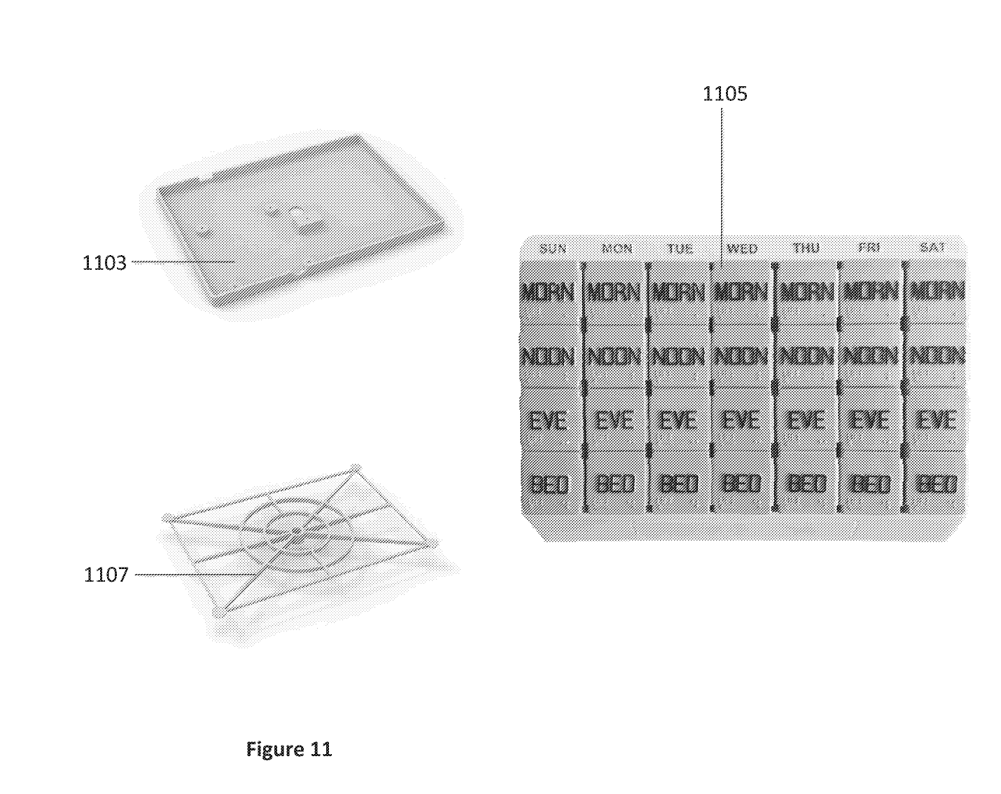

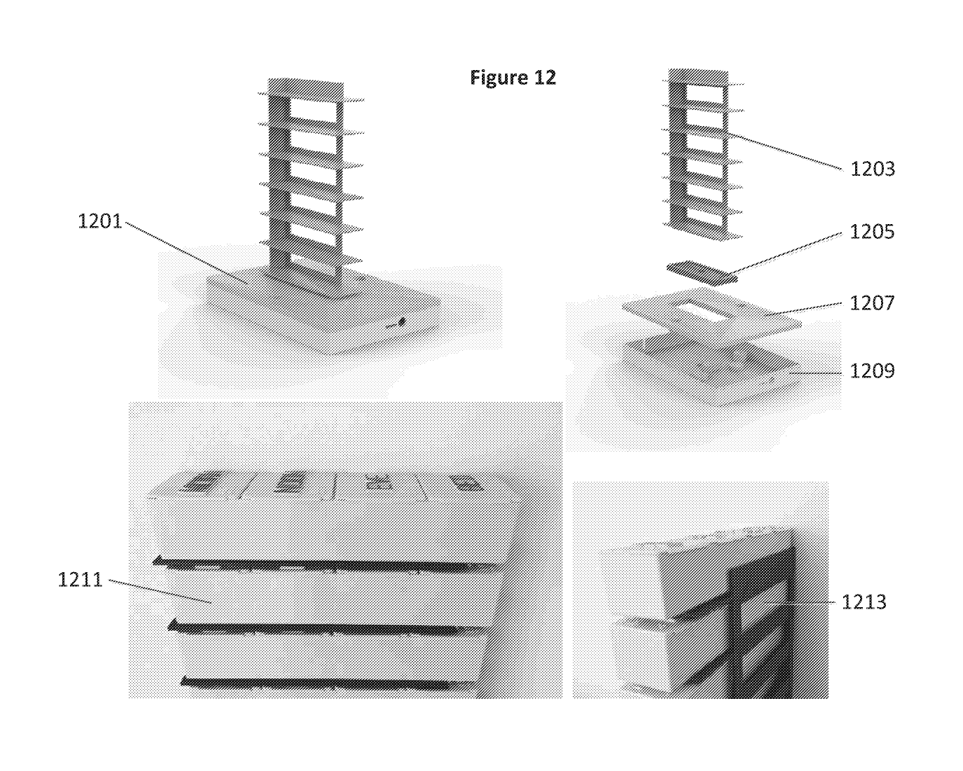

FIG. 11: FIG. 11 shows components for coordinating and assembly with one or more load cells to monitor weight of medications according to one embodiment of the invention. The base 1103 screws onto the bottom 4 corners of the dispenser 1105 and connects to a load cell in the center hole. The tray 1107 holds the board and load cell. The dispensers 1105 shown here include separate compartments for morning, noon, evening, and bedtime medications, in a horizontal 7×4 configuration. This type of dispenser configuration may be suitable for patients with complicated medicinal regimens, particularly where a patient is already familiar with this configuration of dispenser.

FIG. 12: An alternative design for an apparatus that accommodates the dispensers shown in FIG. 11 is illustrated in FIG. 12. This vertical configuration may provide better focus of the force onto a load cell by reducing the torque on the single point of attachment. The same 4-dose dispensers shown in FIG. 11 are loaded onto the shelves of the apparatus. View 1201 shows the fully assembled apparatus. An exploded view of the apparatus shows the tray for holding dispensers 1203, a mounting bracket 1205 for attaching the tray to a load cell (not shown), a cover 1207, and a base 1209 for housing a load cell and a printed circuit board. The apparatus with dispensers added is shown in 1211 (front view) and 1213 (side/back view).

FIG. 13: A device for monitoring adherence to a treatment protocol may be “always on” and therefore susceptible to creep. To account for and address this phenomenon, such a device may follow a process flow as shown in the FIG. 13 flow chart.

FIG. 14: A web application receiving updates from a device for monitoring adherence to a treatment protocol may follow a process flow as shown in the FIG. 14 flow chart.

FIG. 15: An alternative design for an apparatus that accommodates a dispenser similar to the one shown in FIGS. 11 and 12 is shown in FIG. 15. As with FIG. 12, the vertical configuration centers the force on the load cell, minimizing torque. FIG. 15a shows the dispenser 1503 placed in the device 1505. FIG. 15b is an exploded view of FIG. 15a. The dispenser 1503 rests in a receiver 1504 on the device 1505. The top enclosure 1505 and the bottom enclosure 1510 of the device are depicted. A general purpose backlit button 1506 may be present. The load cell 1507, printed circuit board 1508, and a stabilizing weight plate 1509 are all contained within the device. Light pipes 1511 may be used to direct light from LEDs on the bottom of the circuit board to the exterior of the device for visual alerts. Screws 1512 and padded feet 1513 support the device.

### EXAMPLES

Examples of possible embodiments of the present invention are set forth below with reference to the attached figures.

### Example 1—Apparatus

The apparatus device includes a medicine dispenser with at least one container for medicine. Medicine can come in many forms including, but not limited to, pill, liquid, and aerosol. Dispensers may be selected from a variety of configurations, and a simple dispenser and apparatus designed for a single medicine enclosure is shown in FIGS. 1a and 1b. In some embodiments the medicine dispenser may resemble a pre-packaged blister pack. A basic or sophisticated user interface may also be included (not shown).

The dispenser is connected to several components: a scale, controller/processor, memory (temporary and/or permanent), and an input/output controller. If capacitance of the product is measured, the dispenser may be coupled to a clamp or other device for measuring capacitance. Computing components of the apparatus are shown and described in FIGS. 2-3. The scale is sufficiently sensitive and accurate to detect the weight of the medicine involved, including, but not limited to decigrams, centigrams, and micrograms. Or if capacitance is measured, the device for measuring is sufficiently sensitive and accurate to detect changes reflected by removal of a single unit of the product (e.g., a pill). The processor, memory and controllers all connect with the scale either directly or indirectly and manage handling the data. An antenna may be included for connection to the Internet, via Wi-Fi or other wireless data networks. A connector for a direct connection to a computer may also be included, e.g., USB. Lastly, a power source is necessary. The power source can be AC or DC and could be provided via USB, batteries, a wall plug, or other forms sufficient to power this device. Each device has a device unique identification code that is stored in its onboard memory.

The dispenser of the device is filled with the product (e.g., medicine) and the scale or other device takes measurements of its contents at regular intervals. These intervals may take place at specific times during the day, e.g., mid-day and midnight. They may also take place at specific time intervals, e.g., every hour. Or they could take place following certain actions upon the dispenser, e.g., every time it is opened/closed or removed from and returned to the apparatus assembly.

These measurements are transferred to the device's memory and with an available Internet connection; the device may transmit the data immediately. If not transmitted, the data is stored for later transmission, either via the Internet or a direct connection to a computer. The measurement data includes the date-time of the measurement and the measurement. When transmitted, information will also include the device's identification code which links the data and evaluation to the correct patient.

### Example 2—Adherence Monitoring System

Medical professionals prescribe medicine to patients; where the prescription consists of the type of medicine, the amount (dosage), the frequency (the number of doses per a certain amount of time), and perhaps the time: following a certain action, like eating a meal, or at mid-day every day. This prescription is part of a medicinal regimen. A regimen involves the prescription of at least one medication, but may include many more. The medicine in a patient's regimen may be formally prescribed by a physician, recommended by a caregiver (e.g., a daily aspirin), or chosen by the patient (e.g. a daily multi-vitamin).

In the described process, a medicinal regimen is contained within a storage device that measures the weight of its contents at regular intervals. Each time the patient removes medication from this device, the next weight measure will be reduced by the weight of the medication the patient removed. This device may contain one medication from the regimen or many, depending on the goal of monitoring the patient's adherence to a regimen.

The timeline of this weight data is stored for later transmission or transmitted real-time to a receiving application or device, e.g. an internet application with a database. Weights of individual doses/pills have been recorded in this receiving application. Additionally, details of the patient's medicinal regimen have been recorded here. Using the individual medicine weights and the patient's medicinal regimen, the expected remaining weight can be determined.

An example would be a ten-day regimen of penicillin, taken once a day before 12:00 PM. Weight of the prescribed pills is recorded as 500 mg. A ten-day regimen can be determined to weigh 5000 mg (500 mg×10). With the knowledge of the patient's regimen, we can calculate the remaining weight each day should be the weight from the previous day reduced by 500 mg, e.g. 4500 mg after the first dose. The device, measuring at regular intervals, would also record the window of time the reduction took place. Supposing an hourly recording of the weight and a 10:00 AM weight of 5000 mg and an 11:00 AM weight of 4500 mg, one can conclude the medicine was removed from the device between 10:00 AM and 11:00 AM, which complies with the regimen. If no weight reduction was recorded for a specific day, one can conclude that no medication was taken that day by the patient, which does not comply with the regimen.

The receiving device contains the regimen and weight information ready for interpretation, as a result of user inputs in response to prompts from the user interface, which may be accessed via other computing devices. As shown in the example above, adherence is determined by the data points tracked in this device. Specific actions taken by involved parties will be dependent on the situation. For example, a physician who has prescribed seemingly ineffective blood thinners to a patient that seem not to work could use this process to determine that patient's apparent adherence to the regimen. If the patient has complied with the regimen, the physician may increase the dosage or change medication completely. However, if the patient has not complied, the increase dosage or medication change may pose a danger to the patient if they ever adhered to the prescribed regimen, so the physician could keep the regimen as-is and focus efforts on improving adherence or compliance.

Another example of interpretation could take place automatically through an Internet application, when the weight data is transmitted real-time (i.e., the apparatus or system acts as the monitor). If a patient is not complying with the regimen, the apparatus itself, or the remote receiving/processing application can send a notification to the patient as a reminder to take their medication. If this notification is not acted upon and subsequent readings demonstrate nonadherence, notifications may be escalated to caregivers, professional or other designated parties. FIGS. 4-6 show examples of steps in embodiments of an adherence monitoring system.

### Example 3—Medicine Refill System

Use of the systems described requires data to be captured and stored. After capturing and storing this data, it will be evaluated against a preset baseline weight that will describe the said tolerance and evaluate if the remaining medication level/amount meets the specified weight/tolerance. If the remaining medication level/amount does meet that tolerance then the process will end until the next instance where medication is removed. If the remaining medication falls below that tolerance level, a notification or request will be sent to a pre-identified pharmacy, medical distributor, other designated party or contact as per the input settings.

The prescription will then be refilled without the patient needing to specifically request a refill. Alternatively, the pharmacy or other designated party may generate a request to the patient or another designated party (e.g., family member or caregiver) to confirm that a refill is needed or that someone intends to pick up the refill when it is ready. Once the prescription is refilled the pharmacy/medical distributor will confirm that is has been refilled on a device that will connect with the system, so refills are not filled multiple times.

When the patient receives the prescription refill, the new baseline weight will be set and the process will take place all over again. This system will increase compliance levels for patients who regularly forget to call in prescriptions until after their final dose has been taken. This system will also streamline pharmacy/medicinal distribution sites' operations, as it provides sufficient time to plan for demand and it removes some operational bottlenecks, as said pharmacies/distributors can accurately identify timing of their patient's refills and potential trends contained within said information.

### Example 4—Additional Apparatus Embodiments

Dispensers may be selected from a variety of configurations, and additional examples of dispensers are illustrated in FIGS. 8-12. The dispensers and apparatus shown in FIGS. 8-9 allow separate scheduling and monitoring of adherence for each of numerous medications, while the dispensers shown in FIGS. 10-12 allow monitoring of adherence/compliance for groups of medications all scheduled to be taken together. In some circumstances, a patient may already be accustomed to keeping medications in certain types of dispensers. Using an apparatus adapted to a dispenser that is already familiar to a patient, in combination with a base that includes components such as those described in FIGS. 2-3, may facilitate ease in a transition to using a monitoring apparatus and system.

### Example 5—Preemptive Intervention

As data is collected, both for individuals and across patients, the ability to analyze that data for trends increases. This also creates options to preemptively intervene if a monitor (human or technological) determines that a dose or doses may have been missed in a pattern.

In a simple preemptive example, a 60-year-old patient is on statins for cholesterol and takes them 2 times per day, in the morning and evening. She has 3 grandkids that she visits every other Sunday (leaving at 8:00 AM). The monitor determines that she misses the Sunday morning dose more often than not. This historical data is used to identify the trend, which is relayed back to her care providers. Additionally, the system begins to notify her at 7:30 on Sundays (before she leaves the house) that she should remember to take her morning dose.

### Example 6—Predictive Intervention

In similar fashion to Example 5, accumulated data may enable a care provider to predict the likelihood that a particular patient will adhere to a treatment protocol, particularly when aggregated with other health and demographic data.

In a simple predictive example, a physician is trying to decide whether to prescribe a patient medicine A (which is taken 3 times daily and cannot be missed, but costs $10/day) or medicine B (which is taken once daily and costs $100/day). The patient has participated in the adherence monitoring system already, and his historical adherence data is matched up with the adherence data for demographically/adherence similar patients on medicine A. The likelihood for adherence is evaluated in a low-involvement scenario (e.g., he lives alone and has no caregivers outside of the physician) vs. a high involvement scenario (i.e., spouse and family are nearby and actively participate in care). The likelihood of adherence is reported to the physician along this spectrum and the physician makes a more educated/informed decision whether to prescribe medicine A or B.

### Example 7—System for Monitoring Need for Product Refill

A system for monitoring need for refills is useful for but not limited to medical prescriptions or non-prescription pharmaceuticals. In one example, a regular user of a particular high-end cosmetics product may either purchase the product from exclusive department stores or order it online. When ordered online, it is shipped via mail or delivery service. Some users forget to purchase or order the product until their supply is completely exhausted, leading to inconvenient periods of time when the user is without any of their preferred product.

A system using an apparatus of the invention to measure the weight of the cosmetics product may alleviate this problem. The user first purchases a full container of the product and weighs it on the apparatus (e.g., as shown in FIG. 1a), responding to prompts via a user interface to set a reduction to a specific weight percentage (e.g., 20% of starting weight) as the trigger for sending an automatic refill order online. Confirmation of such an order may be requested from the vendor by sending a message to the user. The user may then receive a refill of the product via delivery service before the first container is completely empty.

### Example 8—Integration with Existing Devices

A device sold as SCRIPTALK® may be integrated with an apparatus of the present invention. This is made by En-Vision America and utilizes barcodes added to prescription bottles (i.e., dispensers) to input information about a particular prescription into the apparatus. The apparatus may then cue a user by verbal instructions.

Even without the particular device described above, a speaker may optionally be included in the apparatus to enable voice commands, for example to tell a user it is time to take a medication. In another example, a speaker may read aloud the words received from another party or from the system itself (e.g., read a text message with instructions to the user).

The PILLGUARD® is sold as a pill-dispensing device designed to prevent the abuse of prescription medications. It dispenses only a single dose at a time according to a prescription schedule. This device could be used in conjunction with an apparatus or system of the present invention to track pain pill usage. This would allow aggregation of the usage data along with the user's other medications.

### Example 9—Customized Alerts

Hierarchical alerts are customizable per patient, per medication, and per adherence condition (e.g., missed dose, taken too much, taken too little).

In one customized example, a male patient is on a diuretic (1 pill 3 times daily), presenting circumstances where a missed dose isn't life threatening whereas taking too much could be life threatening.

Scenario 1—Patient does not take his medication at the prescribed time, and the system notifies him (e.g., via text, phone, or email); 1 hour passes and he still doesn't take it, so the system notifies the home health nurse; 30 more minutes pass and still no response, so the system then notifies his son.

Scenario 2—Patient takes all three of his pills in the morning, which is life threatening, so the system skips over the time frame as well as the first notification to him and immediately notifies his home health nurse and son for prompt intervention.

In circumstances where the apparatus alone has all of the prescription information needed to make these determinations, the apparatus may generate the alerts without remote assistance, and may further alert the user to seek medical attention.

### Example 10—Intermediary Devices

The measuring apparatus may not connect directly to the internet, but instead may have an intermediate device that acts as a bridge to the internet and/or an asynchronous storage device. The intermediate device could act as a hub with which multiple devices communicate.

In one example of an intermediate device, a patient is taking multiple medications that require refrigeration. The devices previously described connect to the internet with an onboard Wi-Fi connection. Refrigerators may act as a Faraday Cage, preventing the Wi-Fi signal from passing through. Rather than having multiple external antennas strung through the door of the refrigerator, one bridge unit may be mounted within the refrigerator with an antenna that is outside of the refrigerator, e.g. a small wire. This antenna may also have an audio or visual alert component, e.g., a beep for a reminder and/or a blinking light.

The devices communicate with the bridge unit via wireless connection, e.g., BlueTooth, and the bridge unit send that information to the appropriate web service. In turn the bridge unit may receive information from a web service and communicate back down to the units, e.g., for additional alert lights.

Monitoring and improving patient adherence with Insulin injections (which must be refrigerated) would be an exemplary use for this embodiment of the apparatus.

In another example, a patient travels to and from work with a 7-day a week dispenser that has 3 dosing slots (AM/LUNCH/PM). AM and PM doses are taken at home and LUNCH doses are taken while away from home. The apparatus may be configured to use a cellular phone as a bridge to the Internet and asynchronous storage in this scenario. If the device were to communicate solely over Wi-Fi, the device would need to be configured for each Wi-Fi network with which the patient would interact. This configuration could prove impossible if more than a home and a work network were needed, but additionally must accommodate different lunch sites which may or may not have open Wi-Fi networks. Additionally, someone who travels often may not have a Wi-Fi connection readily available.

Optimal use of this apparatus would require the patient or an assistant to pair the device with a cellular phone (or phones) via a wireless connection, e.g., BlueTooth, and the cell phone would receive the measurement data from the device. The data may be stored within an application on the cellular phone and/or transmitted up to the appropriate web service. The cellular phone could in turn communicate back down to the devices, e.g., to trigger an audio or visual alert.

As will be appreciated, the foregoing examples are illustrative, and non-limiting, of the many possible embodiments of the present invention.

1. A system for monitoring user adherence to a treatment protocol, comprising:
a. an apparatus comprising an enclosure for holding a product and a measuring apparatus for determining a measured characteristic of the product within the enclosure;
b. a data transmission component for real-time or asynchronous transmission;
c. a data receiving component;
d. a data processing component;
e. an alert transmission component.

2. The system of claim 1, further comprising a component for transmitting feedback to the apparatus.

3. The system of claim 1 or 2, wherein the measured characteristic is weight, volume, or capacitance of a product enclosed in the apparatus, and the system detects a change or lack of change in a measured characteristic.

4. The system of claim 1-3, wherein detection of a lack of change in the measured characteristic over a specified time period generates an alert to a designated party.

5. The system of claim 1-4, wherein detection of a predetermined change in weight, capacitance, or volume generates feedback to the apparatus indicating adherence.

6. The system of claim 1-5, wherein aggregate data from a single user or a plurality of users is analyzed to identify trends in adherence or predict likelihood of adherence.

7. The system of claim 1-6, wherein adherence patterns or trends for a single user or a plurality of users generates a preemptive intervention for the single user.

8. The system of claim 1-7, wherein the empty enclosure weighs more than a threshold weight selected to compensate for load cell drift.

9. The system of claim 1-8, wherein a specified event triggers an automatic tare of the load cell in the measuring apparatus.

10. The system of claim 1-9, wherein the specified event is removal of the enclosure from the measuring apparatus.

11. The system of claim 1-10, wherein the specified event is a change in status of a switch on the measuring apparatus.

12. The system of claim 1-11, wherein the specified event is removal of the enclosure from the measuring apparatus and/or a change in status of a switch on the measuring apparatus.

13. The system of claim 1-12, wherein the specified event is a change in status of a sensor on the measuring apparatus.

14. The system of claim 1-13, wherein the specified event is removal of the enclosure from the measuring apparatus and a change in status of a sensor on the measuring apparatus.

15. Computer readable media for monitoring adherence to a treatment protocol using the system of any of claims 1-14.

16. Computer readable media for monitoring need for a product refill according to the system of any of claims 1-14.

17. A method for monitoring adherence to a treatment protocol, comprising detecting capacitance and/or detecting a change or lack of change in capacitance of a product within an enclosure.

18. A method for monitoring adherence to a treatment protocol, comprising detecting capacitance as the measured characteristic using the system of claim 1-14.

19. A method for monitoring adherence to a treatment protocol, comprising:
entering product information/input via web application or website;
receiving data from a specific remote device regarding a characteristic of the product such as weight or capacitance;
processing the data in comparison with data previously received from the specific device; and
generating an alert to a designated party if the data processing yields a particular predetermined result.
